SEQUENCE LISTING (3 sequences)

SEQ ID NO: 1 - protein, 19 aa, Artificial Sequence
  HK-2453 cationic peptide
KHKHKHKHKGKHKHKHKHK

SEQ ID NO: 2 - protein, 19 aa, Artificial Sequence
  H2K-2689 cationic peptide
KHKHKHKHKGKHKHKHKHK

SEQ ID NO: 3 - protein, 9 aa, Artificial Sequence
  T-C terminal end of lysine core - cationic peptide
HHHHNHHHH

(19) United States
(12) Patent Application Publication
(10) Pub. No.: US 2021/0180089 A1
(43) Pub. Date: Jun. 17, 2021

(54) NANOPARTICLES FOR TRANSFECTION

(71) Applicant: LOXEGEN HOLDINGS PTY LTD, Armadale, Victoria (AU)

(72) Inventors: Andrew VENABLES, Armadale, Victoria (AU); Daniel E. LEVY, Armadale, Victoria (AU)

(21) Appl. No.: 17/267,166

(22) PCT Filed: Aug. 14, 2019
(86) PCT No.: PCT/AU2019/050851

§ 371 Date: Feb. 9, 2021

Related U.S. Application Data
(60) Provisional application No. 62/718,616, filed on Aug. 14, 2018.

(51) Int. Cl. C12N 15/88; A61K 48/00; A61K 9/51

(52) U.S. Cl. CPC C12N 15/88; A61K 9/5146; A61K 9/5169; A61K 48/0041

(57) ABSTRACT

This invention is directed to nanoparticles for delivery of nucleic acids to target cells of interest for transfection and expression. The nanoparticles typically include a complex of a cationic peptide bound to a protective hydrophilic polymer through a chelator. The nucleic acid is held to the complex by ionic interactions with the cationic peptide. The chelator is adapted to allow release of the hydrophilic polymer in a time frame suitable to facilitate transfection with the nanoparticle at the target cell surface.

### FIELD OF THE INVENTION

Nanoparticles formulated with nucleic acids which encode bioactive peptides such as therapeutic enzymes, channels, or receptors. The nanoparticles can include protective hydrophilic polymers that are releasable before the nanoparticles enter a target cell of interest.

### BACKGROUND OF THE INVENTION

There are many disease states wherein therapeutic benefits can be obtained through expression of an active form of a missing or defective peptide/gene in particular cells. For example, the ill-effects of phenylketonuria, sickle cell disease, cystic fibrosis, retinoblastoma, or Tay-Sachs, could be mitigated by transfecting working copies of mutated genes.

Cystic fibrosis (CF) remains the most common lethal recessive genetic disease in Caucasian populations, affecting, an estimated, circa 120,000 in the Organization for Economic Cooperation and Development OECD. Median life expectancy has recently risen to ^{˜}38 years (this is a prediction for birth cohorts), but is accompanied by a high burden of disease and treatment. Current median age of death (i.e., for patients today) is about 30 years. The key factor is the chronic destructive infection of the conducting airways in the lung.

In order to understand the CF therapeutic market it is critical to appreciate that: (a) there are at least 1900 different mutations which fall into 5 classes; (b) the science of exactly how the CFTR protein affects the flow of water is complex and not entirely understood, nor is the science of how the CFTR protein interacts with other cell functions; and (c) there is no applicable science identifying how to refold a misfolded protein such as the one present in CF patients. These three points present considerable challenges to the development of small molecule therapeutics. CF is also a challenge to treatment by gene therapy, which has thus far been commercially unsuccessful. For example, there are problems with transfection vectors being cleared by the reticuloendothelial system (RES), and with viscous body fluids restricting diffusion of vectors to target cells of interest.

Current treatments for CF include physical interventions aimed at removing buildup of mucus that clogs airways and creates an environment for pathogens to infect the lungs of patients with CF. For example, patients may spend long periods each day lying face down, receiving chest percussion to prompt movement of mucus out of the lung. Movement of the mucus can also be expedited, e.g., using mucolytics and/or DNAses to break down part of the mucus thickening matrix or use of bronchodilators. Use of antibiotics is important to stave off infections. Finally, a lung transplant may be called for in advanced cases.

In view of the above, a need exists for a gene transfection system that could correct defects in particular cells. It would be desirable to have transfection vectors that are stable in body fluids and capable of readily diffusing to the cell surface of a target cell. The present invention provides these and other features that will be apparent upon review of the following.

### SUMMARY OF THE INVENTION

In a first aspect of the invention, there is provided a complex comprising:

an unnatural cationic peptide comprising a majority of at least two different amino acids selected from the group consisting of: histidine (H) and at least one of: 2,3-diaminopropionic acid, 2,4-diaminobutyric acid, ornithine, and lysine (K); and

a nucleic acid associated with the cationic peptide of the complex through ionic interactions, wherein the nucleic acid encodes an active gene useful for gene therapy; and

optionally, an unnatural hydrophilic polymer further comprising a covalently linked chelating moiety coordinating a metal ion, wherein the cationic peptide additionally coordinates to the metal ion.

A plurality of complexes form nanoparticles, wherein the nanoparticles can function as a gene transfection system for delivering the nucleic acid to a cell. Preferred nanoparticles of the invention provide a transfection vector that is stable in body fluids and/or is capable of readily diffusing to the cell surface of a target cell. The ionic interactions are between positive charges on the cationic peptide and negative charges on the nucleic acid. Suitably, the nucleic acid is one capable of encoding an active gene useful for gene therapy, e.g., plasmid DNA, messenger RNA and the like. In one embodiment, the nucleic acid is not siRNA. It will be understood that in terms of base pairs and size, a nucleic acid is one capable of encoding an active gene useful for gene therapy and is significantly larger than for example, siRNA.

In a second aspect, there is provided a nanoparticle for transfection of a nucleic acid, the

nanoparticle comprising:

a) a complex comprising:
an unnatural hydrophilic polymer bonded to a chelator moiety;
a metal ion chelated to the chelator; and,
an unnatural cationic peptide comprising a majority of at least two different amino acids selected from the group consisting of: histidine (H) and at least one of: 2,3-diaminopropionic acid, 2,4-diaminobutyric acid, ornithine, and lysine (K);

b) a nucleic acid encapsulated into the complex or electrostatically formed in the complex;

wherein the nucleic acid encodes an active gene useful for gene therapy. For example, the metal ion may be a divalent or di-cation chelated to the chelator, for example, Ca²⁺, Zn²⁺, Mg²⁺, Ni²⁺, Cu²⁺, Fe²⁺, and Co²⁺. In some cases the metal ion may be a tri-cation, for example, Fe³⁺ coordinates to a metal ion selected from Ca²⁺, Zn²⁺, Mg²⁺, Ni²⁺, Cu²⁺, Fe²⁺, Fe³⁺ and Co²⁺.

In a third aspect of the invention, there is provided nanoparticles for transfection of a cell with a nucleic acid, the nanoparticles comprising:

an unnatural cationic peptide comprising a majority of at least two different amino acids selected from the group consisting of: histidine (H) and at least one of: 2,3-diaminopropionic acid, 2,4-diaminobutyric acid, ornithine, and lysine (K); and

a nucleic acid associated with the cationic peptide through ionic interactions; and

optionally, an unnatural hydrophilic polymer further comprising a covalently linked chelating moiety coordinated to a metal ion and wherein the cationic peptide additionally coordinates to the metal ion,

wherein the nucleic acid encodes an active gene useful for gene therapy. Preferably, the nucleic acid is plasmid DNA or mRNA capable of encoding an active gene useful for gene therapy.

In a fourth aspect of the invention, there is provided nanoparticles for transfection of a cell with a nucleic acid, the nanoparticles comprising:

an unnatural cationic peptide comprising a majority of at least two different amino acids selected from the group consisting of: histidine (H) and at least one of: 2,3-diaminopropionic acid, 2,4-diaminobutyric acid, ornithine, and lysine (K); and

a nucleic acid associated with the cationic peptide through ionic interactions; and

an unnatural hydrophilic polymer further comprising a covalently linked chelating moiety coordinated to a metal ion and wherein the cationic peptide additionally coordinates to the metal ion,

wherein the nucleic acid encodes an active gene useful for gene therapy. Preferably, the nucleic acid is plasmid DNA or mRNA capable of encoding an active gene useful for gene therapy. The hydrophilic polymer may form a protective layer around the cationic peptide-nucleic acid to disguise the particles against active and passive immune detection and/or to stabilize the nanoparticles from agglomeration in high ionic strength environments, for example, demonstrated by preventing aggregation in 50 mM NaCl for at least 3 hours.

Preferably, the cationic peptides have a sequence selected from I to XI as shown in Table 1 below. Most preferably, the cationic peptides have a sequence selected from: (H-Orn-His-Orn-His-His-Orn-His-His-Orn-His-His-Orn-His-His-Orn-His-His-Orn-His-Orn)₄-Lys-Lys-Lys-His-His-His-His-Asn-His-His-His-His-OH; (H-Lys-His-Lys-His-Lys-His-Lys-His-Lys-His-Lys-His-Lys-His-Lys-His-Lys-His-His-Lys)₄-Lys-Lys-Lys-His-His-His-His-Asn-His-His-His-His-OH; or (H-Lys-His-Lys-His-His-Lys-His-Lys-His-His-Lys-His-Lys-His-His-Lys-His-Lys-His-Lys)₄-Lys-Lys-Lys-His-His-His-His-Asn-His-His-His-His-OH.

Preferably, the nanoparticles have an average diameter of from about from about 50 nm to about 200 nm, more preferably from about 70 nm to about 110 nm.

Preferably, the unnatural hydrophilic polymer is selected from PEG and mPEG which is bonded to a chelator selected from iminodiacetic acid (IDA), ethylenediamine, egtazic acid (EGTA), carboxylmethylaspartate (CMA), dimercaptopropanol, nitrilotriacetic acid (NTA), wherein the chelator coordinates to a metal ion selected from Ca²⁺, Zn²⁺, Mg²⁺, Ni²⁺, Cu²⁺, Fe²⁺, Fe³⁺, and Co²⁺.

Preferably, the nucleic acid comprises a CFTR sequence having at least 90% identity to a functional CFTR (cystic fibrosis transmembrane conductance regulator) gene or an A1AT sequence having at least 90% identity to a functional A1AT (alpha-1 antitrypsin) gene.

The present invention includes nanoparticles for transfection of a nucleic acid, methods of their use, and methods for their administration. It will be understood that when used in transfection, a plurality of the nanoparticles act as a carrier of a nucleic acid payload. Preferred nanoparticles generally include a cationic peptide (e.g., rich in H and K) for associating with the nucleic acid payload through ionic/electrostatic interactions, hydrophilic polymer (e.g., PEG) component which incorporates a chelator moiety for coordination to a metal ion whereby the hydrophilic polymer enhances stability of the nanoparticles, and a metal ion to which the chelator (and bonded hydrophilic polymer) and the cationic peptide coordinates. As previously mentioned, the cationic peptide interacts with the nucleic acid through ionic interactions between positive charges on the cationic peptide and negative charges on the nucleic acid.

In one aspect of the invention, the nanoparticle for delivery and transfection of a nucleic acid includes a complex of an unnatural hydrophilic polymer bonded to a chelator moiety, a metal ion chelated to both the chelator and a cationic peptide. A plurality of nanoparticles make up a composition for administration to transfect nucleic acid material to a plurality of cells.

Exemplary cationic peptides comprise at least two different positively charged amino acids or amino acid analogs, such as, histidine (H), 2,3-diaminopropionic acid, 2,4-diaminobutyric acid, ornithine, and lysine (K).

In certain embodiments, the hydrophilic polymer is a polyether polyol entity or derivative, for example, polyethylene glycol, polytetramethylene ether glycol, polypropylene oxide glycol, polybutylene oxide glycol which can be branched or unbranched, preferably polyethylene glycol (PEG) or methoxypoly(ethylene glycol (mPEG) or mixtures thereof. The PEG or mPEG can be linear or branched, and typically has a molecular weight ranging from 1,000 to 20,000 g/mole.

The chelator moiety can be any appropriate chelator, e.g., depending on the metal ion used and desired affinity level of the association/bond between the metal and the chelated components. Preferred chelators are at least bidentate ligands that have two or more lone pairs of electrons suitable for donation (Lewis base) to a suitably acidic metal capable of accepting the electrons (Lewis acid), thereby forming two or more coordinate/dative bonds to the metal centre to form a metal coordination complex. A coordinate or dative bond is a type of bond in which two shared electrons originate from the same atom. The chelator acts as a ligand to coordinate with the metal cation to form a complex ion. In the complex of the invention, the metal ion may have a coordination number of 3 or more. In some embodiments, the coordination number is 3, 4, 5, 6, 7 or 8, giving trigonal planar, tetrahedral or square planar, trigonal bipyramidal, or octahedral geometries.

Typical chelators employed in the nanoparticles can include iminodiacetic acid (IDA), ethylenediamine, egtazic acid (EGTA), carboxylmethylaspartate (CMA), dimercaptopropanol, nitrilotriacetic acid (NTA), and/or the like. In preferred nanoparticles, the hydrophilic polymer is beneficially adapted to be releasably bound to the cationic peptide. For example, under conditions of the local tissue environment or cell surface, the half-life of the bond between the hydrophilic polymer and the cationic peptide will allow adequate diffusion before contact with the cells of interest. For example, the hydrophilic polymer can remain with the nanoparticle until it is time for the remaining components to be received at the cell surface. In some instances, the half-life of the bond between the functionalized hydrophilic polymer chelator and the metal ion in serum at 37° C. is adapted to be between 5 minutes and 2 hours. It will be understood that releasably bound features may be connected to the lability of one or more of the metal-chelator, metal-cationic peptide bonds. Lability is a well understood concept in the field of coordination chemistry as are techniques to experientially evaluate the same. Typically, the lability will correspond to the bond strength. The bond length between the metal centre and the ligand can be one way of looking at the bond strength and lability of a coordinate bond, where a longer bond is considered more labile than a short bond. The size of the metal cation used as the metal centre and the valence of the metal centre will also influence the lability. The presence of forces including intramolecular forces e.g., H-bonding, or π-back bonding depending on the chelator may also have an effect on the lability of the bond.

Desirably, the nanoparticle hydrophilic polymer may include an extracellular binding or targeting ligand. Such a ligand can have an affinity for a feature (e.g., receptor, membrane protein, etc.) on the surface of a target cell to enhance transfection specificity and efficiency. In certain aspects, the extracellular binding ligand may be covalently linked to the hydrophilic polymer. In some embodiments, the hydrophilic polymer comprises a combination of a first hydrophilic polymer moiety comprising a covalently linked extracellular binding ligand and a second hydrophilic polymer moiety which does not comprise an extracellular binding ligand. In such instances, it is preferred that the first hydrophilic polymer comprising the extracellular binding ligand to be longer than the second hydrophilic polymer. In this way, the targeting or binding ligand can have better access to bind with the cell surface feature.

Suitably, the association of the hydrophilic polymer and cationic peptide is facilitated via a shared attachment point. In one embodiment, the center of coordination for the bond between the hydrophilic polymer and cationic peptide is a metal ion, usually a divalent cation. It will be understood that in such an example, the hydrophilic polymer coordinates or associates with a coordination center to which the cationic peptide further coordinates. The type of bonds which can form between the various entities described are well known in the art of coordination chemistry and include, covalent bonds, donor bonds, coordination bonds, ionic bonds etc. Suitable metal ions have empty or partially empty orbitals which can accept electrons from donor atoms on, or can share electrons from suitable atoms on the hydrophilic polymer and/or the cationic peptide, typically O, N or S atoms. For example, the metal ion can be a metal di-cation, such as a transition metal di-cation, for example, Ca⁺², Zn⁺², Mg⁺², Ni⁺², Cu⁺², Fe⁺², Fe³⁺, and Co⁺², and/or the like.

The cationic peptide can be a natural or unnatural peptide with abundant, preferably sequential, positively charged amino acid residues. For example, the amino acids can be selected from the group consisting of: histidine (H), 2,3-diaminopropionic acid, 2,4-diaminobutyric acid, ornithine, arginine (R), asparagine (N), tyrosine (Y), and lysine (K). Preferred cationic peptides include a majority of H and K residues. It is preferred the cationic peptide have a net positive charge. It is preferred that the peptide include at least 5 sequential amino acids having a net positive charge. It is preferred that at least the first 20 to 10 amino acids have at least 50% positively charged, 75%, 90%, 95%, or 100% positively charged amino acids, under physiological conditions.

Exemplary cationic peptides include, e.g. those shown in the Table below:

TABLE 1

| | | | No. | # of | MW |
|---|---|---|---|---|---|
| No. | Names | Sequence | of AA | branches | (KDa) |
| I | | HHHHNHHHHKKK(KHKHHKHHKHHKHHKHHKHH)₄ | | | |
| II | HK | KHKHKHKHKGKHKHKHKHK | 19 | Linear | 2453 |
| III | H2K | KHKHKHKHKGKHKHKHKHK | 20 | Linear | 2689 |
| IV | H2K2b | K(KHKHHKHHKHHKHHKHHKHK)₂ | 43 | 2 branches | 5779 |
| V | H2K3b | KK(KHKHHKHHKHHKHHKHHKHK)₃ | 63 | 3 branches | 8468 |
| VI | H2K4b | KKK(KHKHHKHHKHHKHHKHHKHK)₄ | 83 | 4 branches | 11157 |
| | | see note 3 | | | |
| VII | H3K4b | KKK(KHHHKHHHHKHHHKHHHK)₄ | 71 | 4 branches | 10191 |
| | H3K8b | See Fig. 11 | 178 | 8 branches | 23218 |
| | (+RGD)¹ | | | | |
| VIII | H2K4bT | KKK(KHKHHKHHKHHKHHKHHKHK)₄T | 92 | 4 branches | 12884 |
| | 2070 | See note 2 | | | |
| IX | H3K4BT | KKK(KHHHKHHHKHHHKHHHK)₄T | 80 | 4 branches | 11755 |
| X | 2595 | (H-Orn-His-Orn-His-His-Orn-His-His-Orn-His-His- | | 4 branches | 11849 |
| | | Orn-His-His-Orn-His-His-Orn-His-Orn)₄-Lys-Lys- | | | |
| | | Lys-His-His-His-His-Asn-His-His-His-His-OH | | | |
| XI | 2596 | (H-Lys-His-Lys-His-Lys-His-Lys-His-Lys-His-Lys-His- | | 4 branches | 12226 |
| | 1:1 Lys:His | Lys-His-Lys-His-Lys-His-His-Lys)₄-Lys-Lys-Lys-His- | | | |
| | | His-His-His-Asn-His-His-His-His-OH | | | |
| XII | 2597 | (H-Lys-His-Lys-His-His-Lys-His-Lys-His-His-Lys-His- | | 4 branches | 12262 |
| | 9:11 Lys:His | Lys-His-His-Lys-His-Lys-His-Lys)₄-Lys-Lys-Lys-His- | | | |
| | | His-His-His-Asn-His-His-His-His-OH | | | |

Note 1
(+RGD) and
Note 2
T =HHHHNHHHH are sequences off the C-terminal end of the lysine core. The terminal sequences of the branched polymers are underlined.
Note 3
A tri-lysine core, represented as KKK or Lys-Lys-Lys, has 4 branch points, the branches come off of the tri-lysine core.

See additional sequences for plasmid and peptides in Appendix of priority application U.S. application Ser. No. 62,718616, filed on 14 Aug. 2018, and in particular the sequence of pGM160 plasmid construct 1, DNA Artificial sequence plasmid construct 2, PRT Artificial sequence Synthetic Construct 3, DNA artificial sequence pGM144 plasmid construct 4, and PRT artificial sequence Synthetic Construct 5, the sequences of which are hereby incorporated by reference.

In certain embodiments the cationic peptides have at least 80%, 90%, 95%, 98% or more identity to peptides listed herein. In many embodiments, branched cationic peptides can provide substantial benefits in packaging and transfection efficiency.

The nucleic acid of the nanoparticle can be any nucleic acid, natural or unnatural, preferably capable of expressing a bioactive peptide. For example, luciferase DNA and GFP expressing DNA have been used herein in experiments. In particular, preferred nucleic acids can encode enzymes, receptors, ion channels, ligands, structural proteins, hemoglobin, and/or the like. The nucleic acid can be a DNA or RNA. The nucleic acid may beneficially be an expression vector, e.g., a DNA plasmid, preferably, including an appropriate promoter. In certain embodiments nucleic acid encapsulated into the capsule/complex of the nanoparticle can have a CFTR sequence having at least 90% identity to the CFTR wild-type CFTR gene. Exemplary known DNA plasmids (including appropriate promoter) include well known pGM160, pGM169, pCF1-CFTR plasmid constructs. These and other DNA plasmids (including appropriate promoter etc. are described in PCT/GB2007/001104 the entire contents of which are herein incorporated by reference. In particular, PCT/GB2007/001104 exemplifies DNA constructs as SEQ ID NO: 1 (pGM160), and SEQ ID NO: 2 (pGM151) on page 3 thereof. Other suitable plasmids include pd1GL3-RL, pBAL and pBACH plasmid DNA, pUMVC-nt-β-gal, pcDNA3.1 WT-CFTR, and pEGFP WT-CFTR as described in J. S. Suk et al./Journal of Controlled Release 178 (2014) 8-17.

The nanoparticle can be any size appropriate for the nucleic acid to be expressed, method of administration, and environment of the target cells. Typically, preferred nanoparticles range in average diameter from about 50 nm to about 200 nm, or from about 70 nm to about 110 nm. In certain instances, the nanoparticle is configured to penetrate cystic fibrosis mucus, e.g., by having a relatively small size (e.g., about 70 nm to 120 nm,) and/or by including a hydrophilic polymer outer coat. The nanoparticles can be adapted for administration to tissue surfaces or within tissues. In many cases it is not desirable to administer by intravenous injection (e.g., due to clearance by the reticuloendothelial system). However, the nanoparticles are typically well adapted for administration on a mucus membrane, intranasally, bronchially intramuscularly, by subdermal injection, by trans-derma injection, by topical application, on an ocular surface, intra-ocular injection, intrathecally, or on a synovial surface. For the respiratory tract, administration can be by inhalation, e.g., using a nebulizer, such as a jet, ultrasonic, or vibrating mesh nebulizer. Of further note is the fact that lipid based vector systems cannot be delivered by vibrating mesh nebulisers due to their viscous nature.

Methods of delivering a nucleic acid to a cell at a mucus membrane are inventive aspects of the nanoparticles. The methods of administration include preparing the nanoparticle (as described herein) and administering nanoparticles to make contact with the desired target cells. A preferred nanoparticle includes a hydrophilic polymer, chelator, metal ion, cationic peptide, and bioactive peptide-encoding nucleic acid. In many embodiments, the hydrophilic polymer comprises PEG or mPEG. The chelator moiety is often an iminodiacetic acid (IDA) or ethylenediaminetetraacetic add (EDTA). The metal ion is often Ca⁺² or Zn⁺². The cationic peptide may comprise at least one of the sequences I to XII described above. The nucleic acid can be any encoding for a useful peptide, e.g., having at least 90% identity to a known CFTR sequence or having at least 90% identity to a known CFTR sequence. The nanoparticles can be administered by any method appropriate to delivery to the desired cell target, e.g., administration by injection, or by inhalation of the nanoparticles in a wet or dry formulation.

In a fifth aspect of the invention, there is provided a method of manufacturing nanoparticles comprising the steps of:

(i) combining a nucleic acid and a cationic peptide to form nucleic acid bearing nanoparticles;

(ii) adding to the nanoparticles in solution, a hydrophilic polymer functionalized with a chelating group chelated to a chelatable metal ion.

The step of combining the nucleic acid bearing nanoparticles and the chelated metal forms adapted nucleic acid bearing nanoparticles in which the ionic charges on the nucleic acid and cationic peptide are insulated or shielded. For example, it is thought that the hydrophilic polymer forms a shell or coating around each nucleic acid bearing nanoparticle. The thus protected nanoparticle may be transported to or migrate to a therapeutic delivery site or surface. This may be an advantage particularly where the nanoparticles are for delivering nucleic acid to target cell or region via topical administration, for example, through pulmonary delivery via nebulization for example. The method may further include the step of controlled the average particle size of the nanoparticles by controlling the pH of the solution. In one embodiment, the pH should be in the range of from about 4.5 to about 7.5. Where inclusion of the hydrophilic polymer is required, the pH is of the solution is preferable from about 6.5 to about 7.5.

Suitably, the method may further comprise the steps of:

a). reacting a hydrophilic polymer with a suitable reactive group on a chelator to covalently bond the chelator moiety to the hydrophilic polymer to form a polymer functionalized chelator; and

b). chelating a metal ion to the polymer functionalized chelator in solution to form a metal complex having a metal ion coordinated by the chelator. It will be understood that where required, reacting and chelating steps occurs before step (ii) above.

In a sixth aspect of the invention, there is provided a use of nanoparticles according to the invention in the manufacture of a medicament for the treatment and/or alleviations of symptoms of a disease or condition requiring topical delivery of a nucleic acid encoding an active gene useful for gene therapy. For example, such use may be in the treatment of one or more of cystic fibrosis and lung disease.

In a seventh aspect of the invention, there is provided a use of nanoparticles according to the invention to transfect a cell with a nucleic acid, preferably wherein the cell is a bronchial cell, particularly an in vivo bronchial cell.

In an eighth aspect of the invention, there is provided a method of treating and/or alleviating the symptoms of one or more of cystic fibrosis, lung disease and liver disease, comprising the step of administering to a subject in need thereof, by delivering in vivo, a therapeutically effective amount of a nucleic acid which encodes an active gene useful for gene therapy against one or more of cystic fibrosis, lung disease and liver disease using nanoparticles of the first aspect as a non-viral transfection agent.

In a ninth aspect of the invention, there is provided a method of delivering a nucleic acid to cell at a mucus membrane, the method comprising:

providing nanoparticles of the first aspect; and,

administering the nanoparticles topically to the mucus membrane. For example, the topical application may involve nebulization of the nanoparticles into the airway. The nanoparticles may be provided in a suitable carrier, for example, a physiological acceptable buffer such as PBS, HEPES, saline, lactated ringers, ultrapure water, and the like.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” can include plural referents unless the content clearly dictates otherwise. Thus, for example, reference to “a surface” can include a combination of two or more surfaces; reference to “bacteria” can include mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein a “nanoparticle” is a particle having dimensions in the nano-range. That is, particles from 1 nanometer (nm) to 1000 nm are nanoparticles. The dimension is in average particle diameter, unless otherwise indicated. Preferred nanoparticles for use in the present invention are typically large enough to contain a nucleic acid of interest and small enough, e.g., to diffuse though intervening biologic fluids to contact a cell of interest for transfection. Typical nanoparticles of the invention range in average diameter from about 50 nm to about 250 nm, preferably from about 50 nm to about 200 nm, more preferably from about 70 nm to 150 nm, most preferably from about 90 nm to 110 nm. In one preferred embodiment, the average diameter is about 120 nm.

A “hydrophilic polymer” is as understood in the art. For example, a hydrophilic polymer typically has adequate amounts of polar and/or ionic groups to be soluble in water (e.g., greater than 1 mg/ml) or wettable with water so that the polymer in dry form absorbs water. It is preferred that the hydrophilic polymer not have substantial hydrophobic qualities (e.g., significant amounts of hydrophobic monomer members), e.g., that would cause the polymer to adsorb significantly onto hydrophobic surfaces.

A “cationic peptide” is a peptide with a net positive charge under physiologic conditions (e.g., at pH 7.4). In the nanoparticles, the cationic peptides typically have no negatively charged amino acids (but for, perhaps the carboxy terminus), 5-fold, 10-fold, or more positive charges than negative. Preferred cationic peptides may include at least one region of at least 10 consecutive amino acids which may have at least 7, 8, 9 or 10 positively charged amino acids depending on the pH of the local environment.

A hydrophilic polymer is “releasably bound” when the bond (e.g., chelation) has a half-life in physiological conditions (pH 7.4, 37° C.) ranging from 30 minutes to 8 hours.

A “ligand” as used herein, refers to a molecule or portion of a molecule that specifically binds to a site, such as a receptor on a target protein.

A “HK rich peptide” as described is a cationic peptide which comprises predominantly the amino acids histidine, lysine and/or lysine derivatives such as ornithine, 2,3-diaminopropionic acid and 2,4-diaminobutyric acid. Arginine (R), asparagine (N) and tyrosine (Y), may also be included.

### BRIEF DESCRIPTION OF THE DRAWINGS

FIG. 1 is a schematic diagram of alternate defects that may lead to cystic fibrosis disease.

FIG. 2 is a chart showing transfection of epithelial cells with nanoparticles having releasably bound hydrophilic polymer. Nanoparticle mediated DNA transfection in BEAS-2B cells (48 hr) Human bronchial epithelial (BEAS-2B) cells were transfected with luciferase DNA formulated in Lipofectamine-2000 (Lipo) or in the forms of nanoparticle without PEG (LG15HKD) or with PEG (LG15HKD-p50) in the presence of 10% FBS (left) or absence of FBS (right) for 4 hour. The transfection was conducted with serial dilution of DNA concentration in triplicated wells. The transfection medium was replaced with regular culture medium and cells were incubated for 48 hours. The transfected cells were lysed and luciferase activity in each well was measured, and normalized against that in cells transfected with 0.05 μg DNA-lipofectamine-2000. Data were represented as the average value from two independent experiments.

FIG. 3 is a chart showing transfection efficiency using nanoparticles with and without PEG, and with and without FBS in the culture media. Nano-particle mediated DNA transfection in BEAS-2B cells (72 hr) Human bronchial epithelial (BEAS-2B) cells were transfected with luciferase DNA formulated in Lipofectamine-2000 (Lipo) or in the forms of nano-particle without PEG (LG15HKD) or with PEG (LG15HKD-p50) in the presence of 10% FBS (left) or absence of FBS (right) for 4 hours. The transfection was conducted with serial dilution of DNA concentration in triplicated wells. The transfection medium was replaced with regular culture medium and cells were incubated for 72 hours. The transfected cells were lysed and luciferase activity in each well was measured, and normalized against that in cells transfected with 0.05 μg DNA-lipofectamine-2000.

FIG. 4 is a chart showing transfection efficiency five days after transfection. Human bronchial epithelial (BEAS-2B) cells were transfected with luciferase DNA formulated in Lipofectamine-2000 (Lipo) or in the forms of nanoparticle without PEG (LG15HKD) or with PEG (LG15HKD-p50) in the presence of 10% FBS (left) or absence of FBS (right) for 4 hour. The transfection was conducted with serial dilution of DNA concentration in triplicated wells. The transfection medium was replaced with regular culture medium and cells were incubated for 5 days. The transfected cells were lysed and luciferase activity in each well was measured, and normalized against that in cells transfected with 0.05 μg DNA-lipofectamine-2000.

FIG. 5 is a chart showing the results of nanoparticle mediated DNA transfection in BEAS-2B cells (48 hr). Human bronchil epithelial (BEAS-2B) cells were transfected with luciferase DNA formulated in Trans-Hi (0.025 ug/well) or in the forms of nanoparticle without PEG or with PEG in the presence or absence of 10% FBS for 5 hour. The transfection was conducted with serial dilution of DNA concentration in triplicated wells. The transfection medium was replaced with regular culture medium at 5 hr post transfection and cells were incubated for 48 hours. The transfected cells were lysed and luciferase activity in each well was measured. Data were represented as the average value from two independent experiments (except F1 one which from only one study).

FIG. 6 is a chart showing the results of nanoparticle mediated DNA transfection in BEAS-2B cells (48 hr). Human bronchil epithelial (BEAS-2B) cells were transfected with luciferase DNA formulated in Trans-Hi (0.025 ug/well) or in the forms of nanoparticle without PEG or with PEG in the presence or absence of 10% FBS for 5 hour. The transfection was conducted with serial dilution of DNA concentration in triplicated wells. The transfection medium was replaced with regular culture medium at 5hr post transfection and cells were incubated for 48 hours. The transfected cells were lysed and luciferase activity in each well was measured.

Data were represented as the average value from two independent experiments (except F1 one which from only one study).

FIG. 7 is a chart showing FACS results in frames a, b and c. Frame a shows a negative control population of cells with no transfection. Frame b shows transfection results for cells treated with pGFP using PEGylated nanoparticles (5 μg DNA/well). Frame c shows cells treated with pGFP/Lipofectamine. M1 represents the population of transfected cells compared to the non-transfected cells represented in Frame a. Frame b shows that the nanoparticle formulation effected transfection in 43.8% of the cells.

FIG. 8 is a chart showing the results of a FACS assay at 72 hours, in terms of the percentage of cells transfected.

FIG. 9 is a chart showing the results of a GFP FACS assay at 72 hours, measuring the degree of GFP activity.

FIG. 10 is a schematic drawing of an exemplary cationic peptide and a self-assembly stage of nanoparticle production.

FIG. 11 is schematic diagram of the 8 branched H3K8b(+RGD) where R=HHHKHHHKHHHK—HHH. The three solid circles connected by a solid line represent the 3-lysine core and K represents the lysine with which the two-terminal branches are conjugated (see Leng at al., Drug News Perspect 20(2), March 2007, pg. 77-86 (‘Mixon’), the content of which is hereby incorporated by reference, particularly Table II and FIG. 6C therein).

### DETAILED DESCRIPTION

The present inventions are directed to certain nanoparticles adapted to transfect cells, and methods of their manufacture and use. The nanoparticles generally comprise a capsule complex and a nucleic acid encoding a bioactive peptide. The complex typically comprises a hydrophilic polymer associated with and/or bound to a cationic peptide to capture, protect, and deliver the nucleic acid. The nanoparticles can be delivered to target cells for transfection by methods of administration including, e.g., localized topical application or an injection. Methods of manufacture include, e.g., Fmoc fabrication of the cationic peptide on a solid support, covalent binding of a chelator to the hydrophilic polymer, charging of the chelator with a divalent metal cation, and (reversibly) binding the hydrophilic polymer to the cationic peptide by interaction with the chelated divalent metal cation.

A number of methods and compositions are discussed in the Summary of the Invention and further details are provided herein and in the Examples section. As would be readily appreciated by the skilled person, the disclosures can be read in combination.

As explained above, preferred nanoparticles may comprise a cationic peptide (e.g., rich in H and K) component which associates with a nucleic acid, a chelator moiety bonded to hydrophilic polymer (e.g., PEG) and a metal ion to which the cationic peptide and the chelator coordinate. Combining the nucleic acid and the cationic peptide forms nanoparticles. Combining the nanoparticles with a metal complex of the metal ion and hydrophilic functionalized chelator results in the formation of a shell of the hydrophilic polymer around the nanoparticles.

The nanoparticles useful for transfection of cells generally include a nucleic acid, preferably plasmid DNA or mRNA, for transfection which is associated with cationic peptide. Preferred nanoparticles are insulated or covered in a shell of protective hydrophilic polymer. The hydrophilic polymer functions in providing stability to the nanoparticles (in vivo, in vitro, and/or in storage and/or administration e.g., by nebulisation) by forming the protective shell around the nucleic acid and cationic peptide, aids in migration through biologic fluids and matrices, and improving pharmacokinetics. The hydrophilic polymer includes a chelator which allows it to bind to the metal cation. The cationic peptide provides features (e.g., positive charges) that interact to bind the nucleic acid cargo and histidines that interact to bind with the chelated metal cation. The nanoparticle is designed to carry the nucleic acid to a cell surface in an efficient fashion, e.g., penetrating viscous body fluids. Particularly, preferred nanoparticles are small, e.g., in a range of about 100 nm diameter allowing diffusion through pores of viscoelastic biofluid polymers (e.g., mucus). Diffusion of the nanoparticles is also aided by the hydrophilic polymer which has little affinity for polymers found in many biofluids. The hydrophilic polymer can be adapted to be releasable from the cationic peptide, aiding in transfection on reaching the target cell.

The nucleic acid cargo in the nanoparticles is surrounded by the protective hydrophilic polymer shell. Preferably, the hydrophilic polymer is adapted to provide increased product stability in storage, reduced aggregation, reduced capture or interference by body fluids, and enhanced diffusion characteristics in body fluids. It is preferred the hydrophilic polymer be hypo-allergenic and not immuno-stimulating. Typically, the hydrophilic polymer is negatively charged or presents a polar surface. In many cases, the hydrophilic polymer is not a natural polymer, e.g., not a naturally occurring carbohydrate, nucleic acid, or peptide.

In certain embodiments, the hydrophilic polymer is a polyethylene glycol (PEG) molecule. For example, the hydrophilic polymer can be PEG or methoxypolyethylene glycol (mPEG). The PEG can be linear or branched. The molecular weight can range from less than 500 to more than 40,000, from 1000 to 25,000, from 2000 to 15,000, or about 10,000.

In many embodiments, the nanoparticles can be directed to target cells by the means of administration, e.g., physically in an organ or tissue compartment. However, the nanoparticles can be even more specifically directed by features providing specific affinity interactions between the nanoparticle and the target cell surface. For example, the hydrophilic polymer and/or cationic peptide can have a ligand (e.g., extracellular targeting ligand) directly or indirectly attached, e.g., covalently or non-covalently. The ligand can be configured to bind to a target cell receptor, preferably a receptor relatively abundant (or found only) on the target cell of interest. In certain embodiments, the ligand can be bound, e.g., at a free end of the hydrophilic polymer. Preferably, the hydrophilic polymer populating the outside of the nanoparticle includes a first hydrophilic polymer type linked to the extracellular binding ligand and a second hydrophilic polymer type that does not comprise an extracellular binding ligand. It can be preferred that the first hydrophilic polymer type be longer than the second type. It can be preferred that the second type be somewhat more releasable (shorter half-life) than the first type. In practice, the nanoparticles can bind to a specific target cell through the specific ligand. This can happen while the nanoparticle is still fully populated with a complete coat of the hydrophilic polymers, or after much of the hydrophilic polymers have been released from the nanoparticle. The presence of the ligand binding feature can allow the nanoparticle to loiter at the cell surface until enough of the hydrophilic polymer is released for transfection to proceed

The hydrophilic polymer is bound to the cationic peptide through a metal ion jointly coordinating to the chelator associated with the hydrophilic polymer and the amino acid residues of the cationic polymer. The chelator is typically associated with a chain end of the hydrophilic polymer via a covalent bond for example. For example, the hydrophilic polymer can covalently bind to a chelator moiety via reaction between suitably reactive functional groups on both entities. The chelator can coordinate with and capture a metal, e.g., leaving other coordination sites to further interact with suitable groups associated with the cationic peptide. Alternately, a chelator can also be associated (e.g. covalently bonded) with the cationic peptide. Any suitable chelator can be used to provide the bond between the hydrophilic polymer and cationic peptide. Exemplary chelators include, e.g., an iminodiacetic acid (IDA), an ethylenediamine, EGTA, dimercaptopropanol, NTA, DPTA, citrate, an oxalate, a tartrate, and the like. Typically, the chelator in the present nanoparticles is an IDA, EDTA, or NTA.

Any suitable metal ion can be used to interact with the chelator on the hydrophilic polymer and with coordinating groups on the cationic peptide. The metal ions are preferably di-cations or tri-cations. For example, the metal ions can be Ca⁺², Zn⁺², Mg⁺², Ni⁺², Cu⁺², Cd⁺², Fe⁺², Fe⁺³, and Co⁺² . Preferably, the chelated metal ion in the present nanoparticles is a Zn⁺², Fe⁺², Fe³⁺, Mg⁺², or Ca⁺² or combinations thereof.

The cationic peptide is configured to interact with the negatively charged nucleic acid to form nanoparticles and also to coordinate with the chelated metal ion, e.g., associated with the hydrophilic polymer. The cationic peptide will have a net positive charge at a pH of use, typically pH 5 to pH 8, or about pH 7.4. The cationic peptide typically features, or has a contiguous region of at least 10 amino acids of, for example including mostly or exclusively positively charged amino acids depending on the pH of the local environment. For example, preferred cationic peptides range in composition from about 10 to about 70 amino acids, from about 15 to about 50, or about 30 amino acids (in the entire peptide, or in a cationic region of the peptide). Preferred cationic peptides include all or a section of from 100% to about 80% positively charged amino acid residues in a section at least 12 amino acids long. In more preferred embodiments, the cationic peptide comprises about 30 to about 50 consecutive amino acids with at least 90% having a positive charge under physiological conditions. In some embodiments, preferred cationic peptides include a majority of H, and one other amino acid selected from the following group: K, 2,3-diaminopropionic acid, 2,4-diaminobutyric acid, and ornithine, can also be included in the cationic peptides of the invention in some embodiments. In more preferred embodiments, at least 90% of the cationic peptide consists of H and K residues; preferably more H residues than K residues (e.g., about 1/3 K residues and about 2/3 H residues). For example, the cationic peptide can include a cationic region abundant in positively charged amino acids. Other amino acids such as arginine (R), asparagine (N) or tyrosine (Y) can also be included in varying amounts. Amino acid analogues, such as histidine (H), 2,3-diaminopropionic acid, 2,4-diaminobutyric acid, ornithine, can also be included in the cationic peptides of the invention in some embodiments. The cationic peptides are linear or branched. In most applications, there can be benefits to using branched peptides. For example packaging and delivery of the nucleic acid can often be improved using branched cationic peptides. The cationic peptide can include 2, 3, 4, 5 branches or more. The cationic peptides are usually prepared synthetically. This usually involves sequential amino acid synthesis on a solid support, e.g., using Fmoc/t-Boc chemistries.

The chelation bond is preferably adapted to render the hydrophilic polymer releasable in an appropriate time frame under conditions of the nanoparticle administration. For example, where the nanoparticles are administered in the presence of a viscous body fluid, the hydrophilic polymer is designed to stay bound long enough for delivery to a membrane surface of a cell targeted for transfection. Generally, under physiological conditions of ionic strength, temperature, and pH, the hydrophilic polymer and its attachment to the metal ion through the chelator functionality should have a half-life from 5 minutes to about 8 hours or more, from 10 minutes to 4 hours, from 30 minutes to 3 hours, or about 2 hours. The optimal half-life would of course depend on, e.g., the distance the nanoparticles must travel between the point of administration and the target cells, the viscosity of the relevant body fluid, and the pore size of any matrix or membrane the nanoparticles must traverse. The half-life of the chelation bond between the hydrophilic polymer, cationic peptide and the metal ion centre can be influenced by, e.g., the choice of chelator, metal ion, and cationic peptide sequence. For example, whereas a Zn⁺² ion strongly interacts forming longer half-life bonds, the half-life of the bond can be moderated using another ion, such as Ca⁺² which tends to form a more labile coordination bond with the chelator. Where the chelator is of a type coordinating at three sites (tridentate), the half-life can be reduced by electing a chelator coordinating at 2 sites (bidentate). Where the cationic peptide binds strongly with a peptide rich in histidine, the half-life can be reduced by reducing the number or percent H in the region interacting with the metal ion. Each of these techniques can be used in combination. The reverse of the operation can strengthen the chelation and extend the half-life. In some cases the environment around the chelation can affect the half-life. For example, where feasible, the chelation bond half-life can be influenced by the pH, ionic strength, or presence of competing ions in the local environment.

The nanoparticle includes nucleic acids of interest, or a nucleic acid encoding a peptide of interest. For example the nucleic acid can be a biologically active RNA, particularly mRNA or DNA preferably encoding a therapeutic peptide. In certain preferred embodiments, the nucleic acid cargo of the nanoparticle can be a DNA (e.g., plasmid) encoding a peptide, e.g., repairing a defect in a cell. For example, the plasmid may be an expression vector expressing functional peptides, such as cystic fibrosis transmembrane conductance regulator (CFTR), sickle cell hemoglobin, hexosaminidase A (Tay-Sachs disease), phenylalanine hydroxylase (phenylketonuria), and the like.

The assembled nanoparticle can have characteristics that aid in delivery to the surface of cells. The nanoparticle can be configured to have a desired charge, hydrophobicity, size, antigenicity, stability, nucleic acid capacity, and the like. In many cases, the nanoparticle has a size well suited to penetration of biologic fluids and membranes. For example, the nanoparticle can be adapted to effectively diffuse through many biological fluids, such as CSF, cell membranes, connective tissue, synovial fluid, mucus, interstitial fluid, clot, vitreous humour, and the like. In many cases, depending on the target cell environment, adequate penetration can be achieved with an assembled nanoparticle ranging in size from less than 50 nm, or from about 50 nm to about 500 nm, preferably from about 75 nm to about 200 nm, more preferably from about 90 nm to 150 nm, most preferably from about 90 nm to 110 nm. In certain preferred embodiments, the average diameter is about 110 nm or 120 nm. The nanoparticle capacity for nucleic acid cargo/payload can be changed by adjustment of the cationic peptide. This can also affect the size of the nanoparticle. The nucleic acid carrying capacity of the nanoparticle can be generally increased by provision of a longer cationic peptide sequence and/or by provision of branch points in the cationic peptide. The outside surface of the nanoparticle can be made less prone to aggregation, have less affinity to biologic fluid matrices, and be less immunogenic, by choice of the hydrophilic polymer. In a preferred embodiment, PEG and PEG-containing copolymers can be the hydrophilic polymer of the nanoparticles. The PEG can form a protective layer around the nanoparticles and disguise the particles against active and passive immune detection. The protective layer around the cationic peptide-nucleic acid may also stabilize the nanoparticles from agglomeration in a high ionic strength environments, for example, in one embodiment, can prevent aggregation in 50 mM NaCl for at least 3 hours.

### Methods of Transfection with Nanoparticles

The nanoparticles can be manufactured, e.g., by bonding a chelator to a hydrophilic polymer, introducing an appropriate metal ion to the chelator to form a metal complex, then combining a cationic peptide associated with a nucleic acid in the form of nanoparticles to form the hydrophilic polymer coated nanoparticles. In other words, the nanoparticles can be manufactured, e.g., by treating a cationic peptide-nucleic acid nanoparticle complex with a pre-assembled hydrophilic polymer covalently linked to a chelator in the form of a pre-formed metal chelate. The nanoparticles of the invention can be stored in a liquid, frozen, freeze-dried, or dried powder formulation before use. The nanoparticles can be administered to a patient in any suitable fashion, e.g., topical, inhalation, or injection.

The formulated nanoparticles can be administered to the intended cells directly or indirectly. In preferred embodiments, the nanoparticles are physically deposited on the cells or within a short diffusion distance from the cells. Depending on the target cells, it may be beneficial to target the nanoparticles using affinity molecules. For example, the nanoparticle can include a ligand (bound anywhere in the complex) specific to any target cell surface feature. This, e.g., in combination with the physical localization of the nanoparticle on administration, can enhance transfection efficiency in the desired cells.

In one aspect of the invention, the nanoparticle is administered to a mucus membrane. For example, the formulated nanoparticles can be inhaled into the lungs to treat cystic fibrosis by introduction of a functional CFTR gene. The formulation can be inhaled as dry powder particles or as an aerosol of liquid droplets, e.g., of a particle size (e.g., about 3 microns, about 1 micron, or less) which can reach the lower reaches of the air passages and alveoli.

In other instances, the nanoparticles can be applied to the intended cells topically (e.g., in a salve) or injected directly into the tissue comprising the intended cells. For example, the nanoparticles can be injected as a liquid suspension through a needle or catheter to a mucus membrane, intranasal, intrabronchial, intramuscular, intraocular, subdermal, trans-dermal, topical, on an ocular surface, intrathecal, the urinary bladder, or synovial surface

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1—Nanoparticle Overview

Preparation of the subject nanoparticles is accomplished on combination of the nucleic acid, e.g., plasmid DNA (pDNA) with cationic peptide e.g., HK polymer. Due to electrostatic interactions occurring between the negatively charged nucleic acid and the cationic peptide, nanoparticles comprising cationic peptide associated with the nucleic acid spontaneously form. By manipulating the formulation conditions, nanoparticles with a desired diameter, for example, nanoparticles with diameters less than 100 nanometers, can be reproducibly produced. Furthermore, additional of the stabilizing hydrophilic polymers described, e.g. via PEGylation by grafting PEG-chelator to the surface of the nanoparticles provides added stability with no significant impact on overall nanoparticle size.

### Example 2—Development of the Nanoparticle Formulation Method

The inventors established protocols to: a) Prepare sub 140 nm diameter nanoparticles comprising cationic peptide (HK rich peptide designated 2070 in Table 1 above) and pDNA to form HKD nanoparticles (HKplasmidDNA particles); b) PEGylate nanoparticles post formation to form HKDp particles (HKplasmidDNA-PEGylated; c) Test nanoparticle stability in PBS and NaCl. Only PEGylated HKD particles (HKDp) resisted size increase over time and with increasing ionic strength; d) Test the pH range for optimal HKD particle formation.

Preparation of nanoparticles was studied at pH 5 to 8 in 0.5 pH value increments. Particle size and DNA encapsulation was monitored. Optimal particle sizes were achieved between pH 5.0-7.0. Optimal DNA encapsulation was achieved between pH 5.5-7.0. pH 6.5+/−0.5 was selected in order to ensure robust particle formation close to physiological pH.

In cell culture media, HKD particles aggregated within a few minutes. HKDp particles were relatively more stable. In cell culture media with serum, there was an initial size increase to greater than 100 nm with stabilization under 200 nm up to 96 hrs. In cell culture with no serum, HKDp particle diameters remained unchanged during the first 40 minutes. However, after 24 hours, the particle sizes increased more rapidly than noted for particles in serum. Non-PEGylated nanoparticles are determined not stable in PBS, and NaCl solutions while PEGylated nanoparticles show prolonged stability over time and with incrementally increasing ionic strength. Longer stability of the nanoparticle formulations on storage at 2-8° C. requires study.

Nanoparticles (PEGylated and non-PEGylated) were successfully fluorescently labelled using PicoGreen, Propidium Iodide, and Alexa 488 labeling reagents. Labeled particles with diameters less than 100 nm could not be detected using optical microscopes. Very few particles in the range of 150-200 nm and a few more particles greater than 200 nm could be observed. With these observations, it is important to recognize that particles form in a size distribution with larger particles making up a minority of the overall formulation.

Overall, the inventors have developed reproducible methodologies for preparation of HKD particles under 80 nm, and HKDp particles under 100 nm. Along with HKD and HKDp formulation development, the inventors have developed a sensitive and robust Agarose Gel method for monitoring pDNA encapsulation qualitatively, and/or particle stability at various salt concentrations. Also, there was evidence that PEG disassociated from the nanoparticle after 48 to 72 hours, as the inventors had designed it to do.

### Example 3—Transfections with Nano-Particles

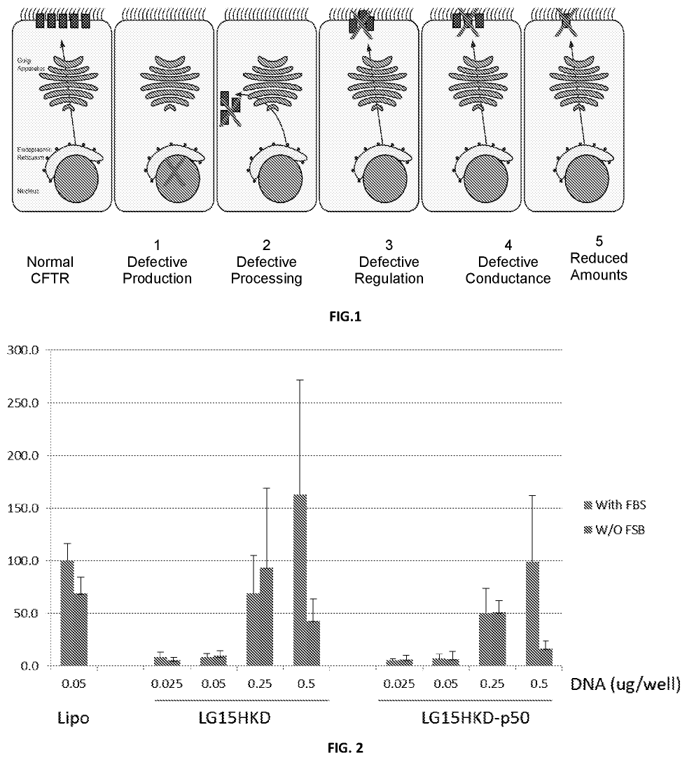

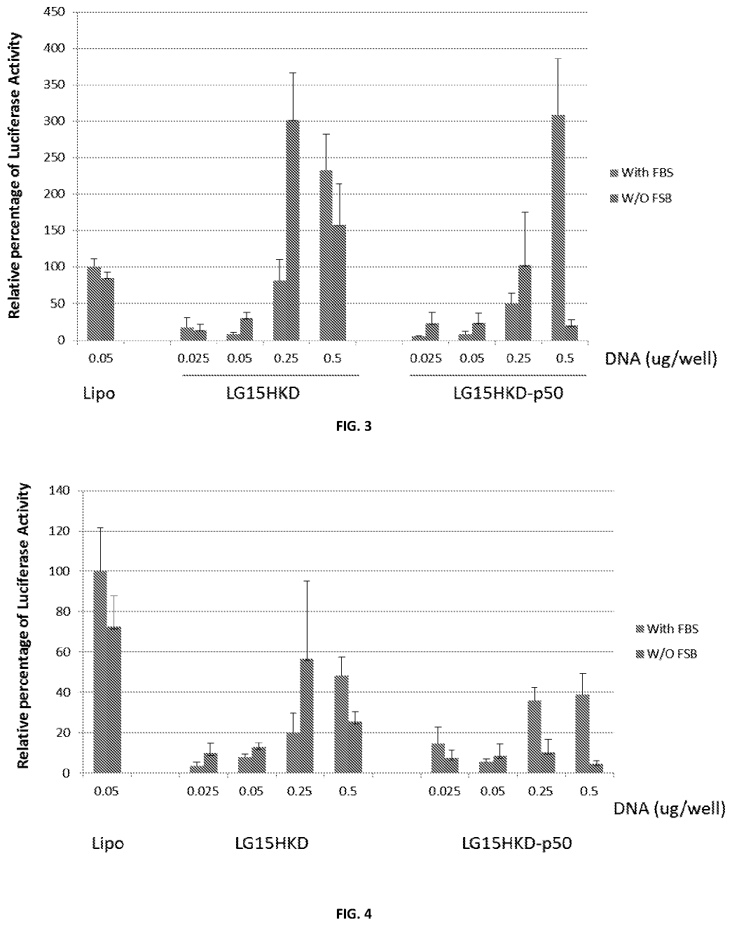

The inventors' nanoparticles are active in mediating DNA transfection in BEAS-2B cells. In three transfection studies conducted 1-week apart, the nanoparticles demonstrated comparable activities in effecting DNA transfection in BEAS-2B cells. In general, at 48 hours, the nanoparticles (LG15HKD)(LG15 is a luciferase plasmidDNA) and LG15HKD-p50 (pegylated nanoparticle) yielded lower luciferase expression than achieved by Lipofectamine-mediated transfection. It is estimated that 5 to 10-fold more DNA is required for the nanoparticles to achieve similar or higher levels of transfection compared to Lipofectamine (see, FIGS. 2 and 3). Overall the LG15HKD was more effective for transfection compared to LGHKD-p50. However, the difference may not be significant (see, FIGS. 2, 3, and 4). The nanoparticles of the invention are well tolerated by BEAS-2B cells with no observed significant cell count reduction as a function of nanoparticle concentration. The low cellular toxicity of the nanoparticles is further confirmed by the linear curve of DNA levels as indicated by luciferase activity (see, FIGS. 2, 3, and 4). This reflects a dose like response.

The nanoparticles mediated DNA transfection with greater efficiency compared to Lipofectamine-mediated at 72 hours (FIG. 2). In contrast, at 120 hours post transfection, transfection efficiency of the nanoparticles was lower compared to Lipofectamine-mediated transfection (FIG. 3). This observation suggests that the nanoparticle transfection may be via a pathway which differs from Lipofeactamine-mediated transfection. It may also be the result of a higher DNA concentration per well.

Repeat experiments using different formulations yield consistent results showing strong transfection. Formulations comprising each of the 4 below peptides and PEG were tested against Luc transfection facilitated by the Trans-Hi transfection agent (similar to lipfectamine). Testing was executed at 3 concentrations (0.1 ug/well, 0.5 ug/well and 1.0 ug/well) in a 96 well plate format. Each peptide formulation was prepared both with and without PEG, and tested accordingly. Generally the formulation with the even number comprises PEG grafted to the nanoparticles (F2 ,F6, F10 and F14). F1 and F2 are reproductions of the original formulations. Formulations—F1: 2070/Luc non-PEG, 50 ug/mL; F2: 2070/Luc-PEG, 47.62 ug/mL; F5: 2595/Luc non-PEG, 50 ug/mL; F6: 2595/Luc-PEG, 48 ug/mL; F9: 2596/Luc non-PEG, 50 ug/mL; F9: 2596/Luc-PEG, 48 ug/mL; F13: 2597/Luc non-PEG, 50 ug/mL; F14: 2597/Luc-PEG, 48 ug/mL; F17: Luc in Hepes, 50 ug/mL, whereby 2070 is (original peptide HK, 2:3 Lys:His in Table 1 above), 2595 is the ornithine peptide X above in Table 1, 2596 is 1:1 Lys:His is peptide XI in Table 1 above, and 2597 is 9:11 Lys:His, peptide XII in Table 1 above.

| | | Average Particle |
|---|---|---|
| Peptide | | Diameter (via ZETA Pals |
| Code | Cationic Peptide Sequence | particle size analysis) |
| 2070 | (H-Lys-His-Lys-His-His-Lys-His-His-Lys- | 58.5 nm (luciferase, non-PEG) |
| HK | His-His-Lys-His-His-Lys-His-His-Lys-His- | 66.0 nm (GFP, non-PEG) |
| 2:3 Lys:His | Lys)4-Lys-Lys-Lys-His-His-His-His-Asn- | 94.5 nm (luciferase, PEG) |
| | His-His-His-His-OH | 127.4 nm (GFP, PEG) |
| 2595 | (H-Orn-His-Orn-His-His-Orn-His-His-Orn- | 65.9 nm (luciferase, non-PEG) |
| 2:3 Orn:His | His-His-Orn-His-His-Orn-His-His-Orn-His- | 73.7 nm (GFP, non-PEG) |
| | Orn)₄-Lys-Lys-Lys-His-His-His-His-Asn- | 70.9 nm (luciferase, PEG) |
| | His-His-His-His-OH | 101.3 nm (GFP, PEG) |
| 2596 | (H-Lys-His-Lys-His-Lys-His-Lys-His-Lys- | 64.3 nm (luciferase, non-PEG) |
| HK | His-Lys-His-Lys-His-Lys-His-Lys-His-His- | 82.1 nm (GFP, non-PEG) |
| 1:1 Lys:His | Lys)₄-Lys-Lys-Lys-His-His-His-His-Asn-His- | 77.4 nm (luciferase, PEG) |
| | His-His-His-OH | 124.1 nm (GFP, PEG) |
| 2597 | (H-Lys-His-Lys-His-His-Lys-His-Lys-His- | 51.3 nm (luciferase, non-PEG) |
| HK | His-Lys-His-Lys-His-His-Lys-His-Lys-His- | 54.7 nm (GFP, non-PEG) |
| 9:11 Lys:His | Lys)₄-Lys-Lys-Lys-His-His-His-His-Asn-His- | 86.0 nm (luciferase, PEG) |
| | His-His-His-OH | 90.9 nm (GFP, PEG) |

Some observations: with the original formulations, consistency with earlier experiments is demonstrated. The PEG formulations perform better than those without. Serum appears to aid transfection in the higher concentration but not so much in the lower concentrations. Transfection is lower in absolute terms at 72 hours but much better in comparative terms to the Trans Hi-Luc formulation—consistent with consideration that it takes time for the PEG to dissociate prior to transfection. It is possible that the PEG dissociated more rapidly for F2, F6, and F10 compared to F14. Weaker transfection for non-PEGylated formulations may be related to inherent nanoparticle instability in the cell culture media. For the most effective formulation to date, transfection efficiency may be up to approx. 700 percent better compared to the Trans-Hi formulation (noting different concentrations). Most important is that Luc without a transfection agent produced no signal. There is an apparent dose-response correlating well concentration to transfection.

### Example 4—Nano-Particle Stability

The nanoparticle formulations are stable for mid-term to long-term storage. The nanoparticles (LG15HKD and LGHKD-p50), stored at 4° C., retained activity during the 3 week period of the 3 transfection studies. However, there is a possibility that these formulations may become less effective in the presence of 10% FBS during the transfection process. In the first transfection study conducted 5 days after nanoparticle preparation, nanoparticles were ineffective for transfection in the presence of 10% FBS. However, in the later transfection studies (11 days and 18 days after nanoparticle preparation), both LG15HKD and LGHKD-p50 remained effective for transfection in the presence of 10% FBS. These results were comparable to parallel studies run in the absence of FBS.

### Example 4—Fluorescently Activated Cell Sorting

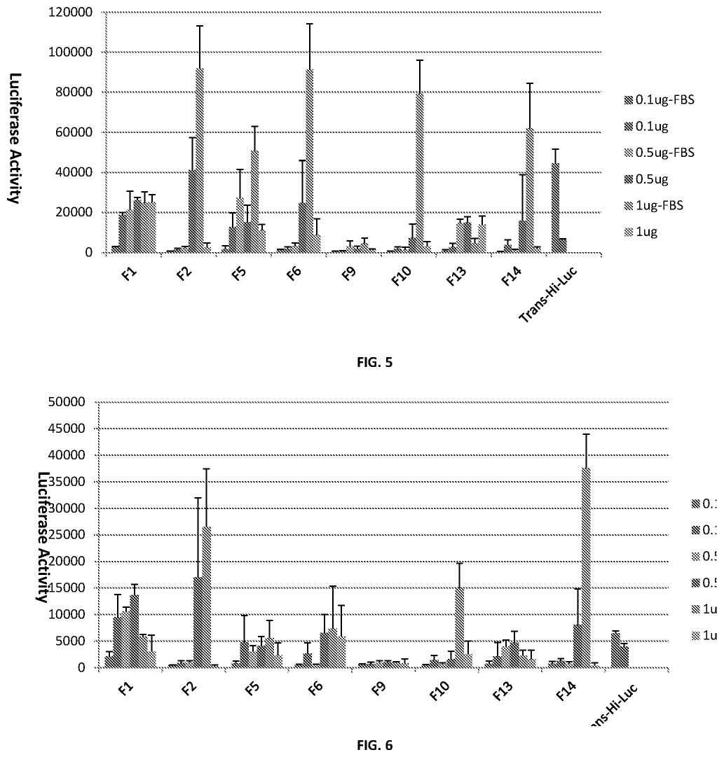

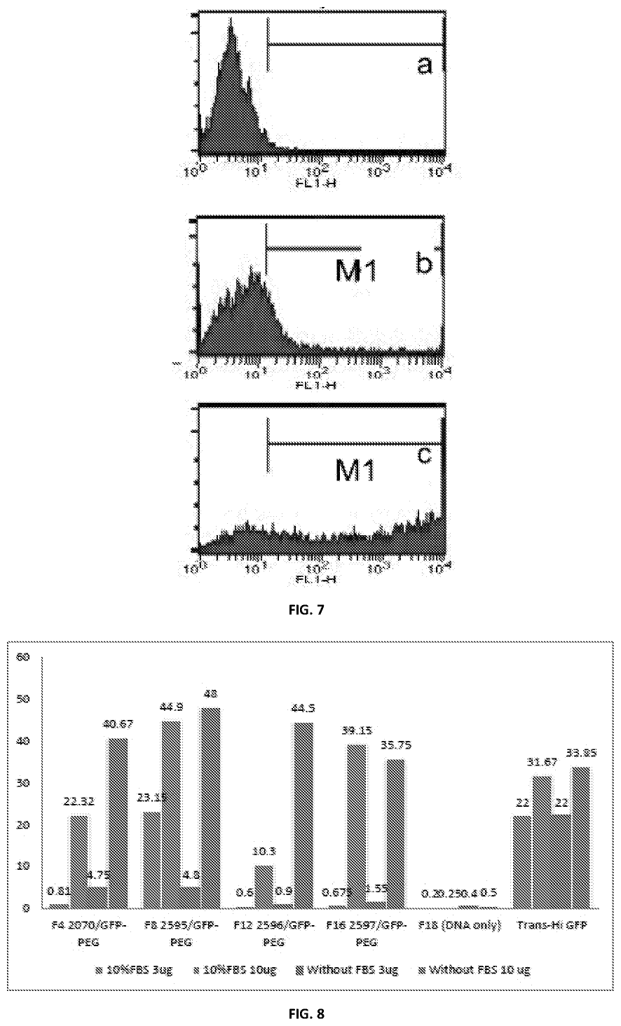

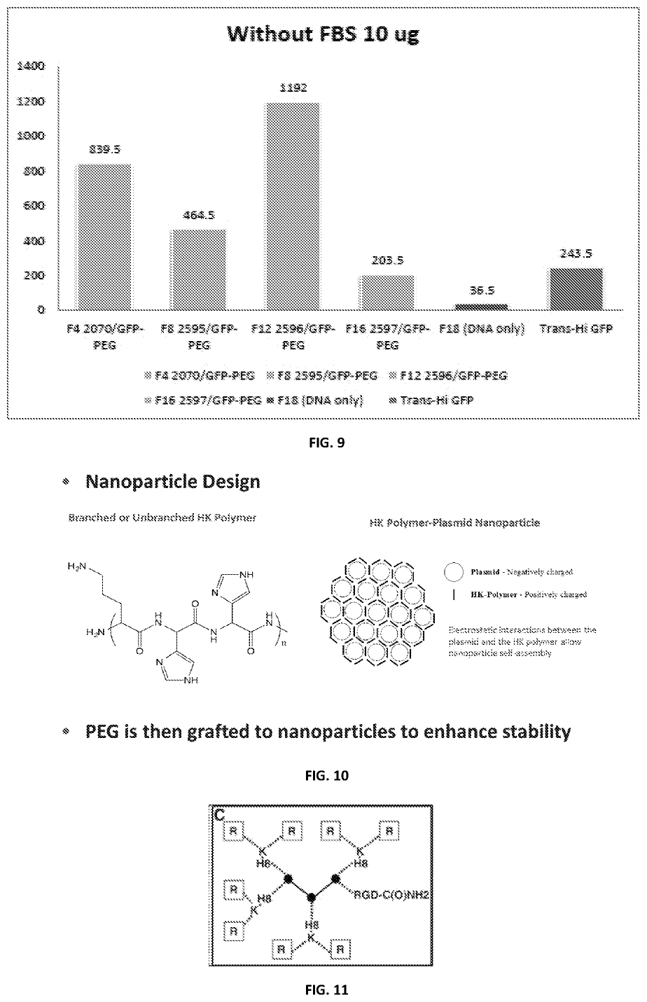

In this experiment, using sophisticated lasers, all of the cells in each well in a plate of wells were counted to see how many cells are transfected. This is a more precise way of measuring the efficacy of the nanoparticles and is an important assay for optimization for maximum stability, size and transfection efficiency. The results indicate transfection in up to 43.8% of cells which, if replicated in a human lung, would deliver a clinically meaningful result. The inventors then conducted a similar experiment using variants of the peptide to show the pathway to optimization. The nanoparticles show GFP activity up to 32 times higher than the control particles, being naked DNA. FIGS. 8 and 9 show the result of transfection using the same variants of the peptides as were used in FIGS. 5 and 6 above. As can be seen the transfection rate improved to up to 48% of cells. In the case of GFP activity, the nanoparticles perform up to 32 times better than the control, being naked DNA.

### Example 5—Protocol for Preparing Peptide/DNA Nanoparticles & PEGylated Nanoparticles

The DNA was diluted to 50-100 ug/mL in 5mM HEPES pH 7.4. A peptide solution at a concentration of 100-180 ug/mL in 5 mM HEPES was prepared. A 250 uL volume of the DNA solution was transferred into a 2 mL Eppendorf tube. Using a 100 uL peptide, an equal volume of the peptide solution was titrated, at an appropriate rate to avoid aggregation, into the DNA solution while vortexing. The solution slowly turns translucent and no visible particles should occur. The particle size is measured using a dynamic light scattering (DLS) instrument (such as the Brookhaven ZetaPALS). The particle size should be below 100 nm, preferably below 80 nm. The nanoparticles were filtered through a 0.22 um sterile filter. The formulation was stored in a refrigerator (2-8 deg C.) until desired for use and/or for PEGylation. Where PEGylated peptide/DNA nanoparticles are required, a PEG-Zn solution was prepared in a separate tube at a concentration of about 400 mg/mL. 25-100 uL of the PEG solution was slowly added to 500 uL of the peptide/DNA nanoparticle preparation, depending on the degree of PEG-coating required. The particle size was measured by DLS. The particle size should be about 100-140 nm depending on the amount of PEG added. The formulation was store in a refrigerator (2-8 deg C.). Above describes a range for the peptide/DNA ratio which can be used in one embodiment. In a preferred example, the ratios used for nanoparticle preparation were: 100 ug/mL DNA and 100 ug/mL peptide mixed at equal volume. PEGylation: 50 uL of 440 mg/mL PEG-IDA-Zn into 500 uL nanoparticles as prepared above.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes

1-36. (canceled)

37. Nanoparticles for transfection of a cell with a nucleic acid, the nanoparticles comprising:
an unnatural cationic peptide comprising a majority of at least two different amino acids selected from the group consisting of: histidine (H) and at least one of: 2,3-diaminopropionic acid, 2,4-diaminobutyric acid, ornithine, and lysine (K); and
a nucleic acid associated with the cationic peptide through ionic interactions; and
wherein the nucleic acid is a functional nucleic acid capable of encoding an active gene useful for gene therapy.

38. Nanoparticles of claim 37, the nanoparticles further comprising: an unnatural hydrophilic polymer bonded to a chelator coordinated to a metal ion and wherein the cationic peptide coordinates to the metal ion.

39. Nanoparticles of claim 37, provided in a physiologically acceptable buffer such as PBS, HEPES, saline, lactated ringers, or ultrapure water.

40. Nanoparticles of claim 37, wherein cationic peptide and the nucleic acid charge:charge ratio is balanced.

41. Nanoparticles of claim 38, wherein the molar ratio of the unnatural hydrophilic polymer bonded to a chelator coordinated to a metal ion and the cationic peptide is greater than 50:1.

42. Nanoparticles of claim 38, wherein the hydrophilic polymer forms a protective layer around the cationic peptide-nucleic acid nanoparticle core and/or wherein the hydrophilic polymer stabilizes the nanoparticles as demonstrated by resistance to agglomeration in a high ionic strength environment, substantially no aggregation in 50 mM NaCl for at least 3 hours.

43. Nanoparticles of claim 37, wherein the nucleic acid is an expression vector expressing functional peptides selected from CFTR, A1AT, sickle cell hemoglobin, hexosaminidase A (Tay-Sachs disease), or phenylalanine hydroxylase (phenylketonuria) or a CFTR sequence having at least 90% identity to a functional CFTR gene or comprises an A1AT sequence having at least 90% identity to a functional A1AT gene.

44. Nanoparticles of claim 37, adapted for topical delivery on a mucus membrane, intranasal, intrabronchial, intramuscular, subdermal, intraocular, trans-dermal, topical, on an ocular surface, intrathecal, or synovial surface.

45. Nanoparticles of claim 37, wherein the nanoparticles has an average diameter ranging from about 50 nm to about 250 nm.

46. Nanoparticles of claim 38, wherein the hydrophilic polymer is PEG or mPEG, wherein the PEG or mPEG is linear or branched.

47. Nanoparticles of claim 37, wherein the chelator moiety is selected from the group consisting of: an iminodiacetic acid (IDA), an ethylenediamine, ethylenediaminetetraacetic acid (EDTA), egtazic acid (EGTA), carboxylmethylaspartate (CMA), dimercaptopropanol, and nitrilotriacetic acid (NTA).

48. Nanoparticles of claim 38, wherein the metal ion is selected from the group consisting of: Ca²⁺, Zn²⁺, Mg²⁺, Ni²⁺, Cu²⁺, Fe²⁺, Fe³⁺ and Co²⁺.

49. Nanoparticles of claim 38, wherein the hydrophilic polymer is adapted to be releasably bound to the cationic peptide and wherein a half-life of a chelation bond between the hydrophilic polymer and cationic peptide in serum at 37° C. is adapted to be between 5 minutes and 8 hours.

50. Nanoparticles of claim 37, wherein the cationic peptide has at least 90% identity with any of the following peptides:

| No. | Names | Sequence |
|---|---|---|
| I | | HHHHNHHHHKKK(KHKHHKHHKHHKHHKHHKHH)₄ |
| II | HK | KHKHKHKHKGKHKHKHKHK |
| III | H2K | KHKHKHKHKGKHKHKHKHK |
| IV | H2K2b | K(KHKHHKHHKHHKHHKHHKHK)₂ |
| V | H2K3b | KK(KHKHHKHHKHHKHHKHHKHK)₃ |
| VI | H2K4b | KKK(KHKHHKHHKHHKHHKHHKHK)₄ |
| | | see note 3 |
| VII | H3K4b | KKK(KHHHKHHHHKHHHKHHHK)₄ |
| | H3K8b | See Fig. 11 |
| | (+RGD) [symbol] | |
| VIII | H2K4bT | KKK(KHKHHKHHKHHKHHKHHKHK)₄T |
| | 2070 | See note 2 |
| IX | H3K4BT | KKK(KHHHKHHHKHHHKHHHK)₄T |
| X | 2595 | (H-Orn-His-Orn-His-His-Orn-His-His- |
| | | Orn-His-His-Orn-His-His-Orn-H [symbol] |
| | | His-Orn-His-Orn)₄-Lys-Lys-Lys-His- |
| | | His-His-His-Asn-His-His-His-His [symbol] |
| | | OH |
| XI | 2596 | (H-Lys-His-Lys-His-Lys-His-Lys-His- |
| | 1:1 | Lys-His-Lys-His-Lys-His-Lys-H [symbol] |
| | Lys:His | Lys-His-His-Lys)₄-Lys-Lys-Lys-His- |
| | | His-His-His-Asn-His-His-His-His- [symbol] |
| | | OH |
| XII | 2597 | (H-Lys-His-Lys-His-His-Lys-His-Lys- |
| | 9:11 | His-His-Lys-His-Lys-His-His-Ly [symbol] |
| | Lys:His | His-Lys-His-Lys)₄-Lys-Lys-Lys-His- |
| | | His-His-His-Asn-His-His-His-His- [symbol] |
| | | OH |

[symbol]

indicates data missing or illegible when filed

51. Nanoparticles of claim 37, wherein the nanoparticle further comprises an extracellular targeting ligand.

52. A method of manufacturing nanoparticles according to claim 38, comprising the steps of:
(i) combining the nucleic acid and the cationic peptide to form nucleic acid bearing nanoparticles;
(ii) adding to the nanoparticles in solution, the hydrophilic polymer functionalized with a chelating group chelated to the chelatable metal ion.

53. The method of claim 52, further comprising the step of controlling the solution pH to vary the nanoparticles average particle diameter.

54. A method of treating and/or alleviating the symptoms of one or more of cystic fibrosis, lung disease and liver disease, comprising the step of administering to a subject in need thereof, a therapeutically effective amount of the nanoparticles as defined in claim 37.

55. A non-viral vector for transfection of a bronchial cell with nucleic acid encoding a CFTR sequence having at least 90% identity to a functional CFTR gene, the vector comprising nanoparticles having an average particle diameter of from about 50 nm to about 250 nm, and including:
unnatural branched cationic peptides comprising a majority of at least two different amino acids selected from the group consisting of: histidine (H) and at least one of: 2,3-diaminopropionic acid, 2,4-diaminobutyric acid, ornithine, and lysine (K); and
a plasmid DNA associated with the cationic peptide through ionic interactions; and
a PEG or mPEG polymer bonded to a chelator coordinated to a Zn²⁺ metal ion and wherein the cationic peptide also coordinates to the Zn²⁺ metal ion, and
wherein the DNA is a plasmid DNA or a mRNA capable of encoding the CFTR sequence.

56. The non-viral vector of claim 55, wherein the plasmid DNA is pGM160, pGM169, pCF1-CFTR, pGM151, pd1GL3-RL, pBAL, pBACH, pUMVC-nt-β-gal, pcDNA3.1 WT-CFTR, pEGFP WT-CFTR, or luciferase plasmid DNA.
